(19) United States
(12) Patent Application Publication
(10) Pub. No.: US 2023/0157555 A1
(43) Pub. Date: May 25, 2023

(54) BLOOD-VOLUME-BASED CUFF-LESS NON-INVASIVE BLOOD PRESSURE MONITORING

(71) Applicant: Shenzhen Goodix Technology Co., Ltd., Shenzhen (CN)

(72) Inventors: Yi He, San Diego, CA (US); Bo PI, San Diego, CA (US)

(21) Appl. No.: 17/535,666

(22) Filed: Nov. 25, 2021

(51) Int. Cl. A61B 5/021; A61B 5/00

(52) U.S. Cl. CPC A61B 5/02141; A61B 5/0082; A61B 5/0017; A61B 2562/164; A61B 2560/0406; A61B 2560/0431; A61B 2560/0238; A61B 2560/0228

(57) ABSTRACT

Techniques are described for non-invasive, cuff-less measurement of blood pressure of a user using a portable electronic device. Illumination is projected through a body part and received by photodetectors on the other side of the body part. The body part includes elastic pathways of the circulatory system through which blood flows. Cycles of contraction and relaxation by the heart cause pulse waves to travel through the blood, which cause volumetric changes in the elastic pathways. The transient changes in blood volume result in corresponding transient changes in the amount of illumination that is absorbed by the body part versus the amount that passes through to the photodetectors, as manifest by a detection output signal. Calibration data can be used to convert the detection output signal to blood pressure measurements, such as including diastolic and systolic blood pressure readings.

### TECHNICAL FIELD

This disclosure relates to portable medical monitoring devices, and, more particularly, to portable electronic devices for blood-volume-based cuff-less non-invasive blood pressure monitoring.

### BACKGROUND

Portable electronic devices with various types of sensors have become ubiquitous. People commonly walk around with smartphones and wearable devices (e.g., fitness trackers, smart watches, etc.) that periodically and/or continuously detect and record sensor data about the person and/or the person's environment. Many such sensors help monitor changes in physical and/or mental state of a user, such as for fitness tracking, biofeedback, etc. For example, many modern smart watches have sensors to monitor a wearer's body temperature (e.g., using a thermocouple), heart rate or pulse (e.g., using optical or ultrasonic reflection), number of steps (e.g., using a pedometer), etc. However, conventional portable electronic devices and techniques have not tended to be able to provide accurate blood pressure measurements.

### SUMMARY

Embodiments provide systems and methods for non-invasive, cuff-less measurement of blood pressure of a user using a portable electronic device. Illumination is projected through a body part and received by photodetectors on the other side of the body part. The body part includes elastic pathways of the circulatory system through which blood flows. Cycles of contraction and relaxation by the heart cause pulse waves to travel through the blood, which cause volumetric changes in the elastic pathways. The transient changes in blood volume result in corresponding transient changes in the amount of illumination that is absorbed by the body part versus the amount that passes through to the photodetectors, as manifest by a detection output signal. Calibration data can be used to convert the detection output signal to blood pressure measurements, such as including diastolic and systolic blood pressure readings.

According to one set of embodiments, a system is provided for non-invasive cuff-less measurement of blood pressure of a user. The system includes: an illumination subsystem to project illumination through a body part that includes at least one elastic blood circulatory pathway through which flows a continuously changing volume of blood, the illumination being at a frequency absorbed by blood; an optical detection subsystem to receive portions of the illumination passing through the body part without absorption or reflection, and to generate a detection output signal based on the received portions of the illumination to correspond to the continuously changing volume of blood; a portable housing by which to hold at least one of the illumination subsystem or the optical detection subsystem against the body part with a holding force during a measurement routine including illumination and optical detection; a pressure detection subsystem integrated with the portable housing to monitor the holding force during the measurement routine; and a control processor to generate a blood pressure output signal based on the detection output signal and calibration data, the calibration data associating blood volume to blood pressure for the user and accounting for the holding force.

According to another set of embodiments, a method is provided for non-invasive cuff-less measurement of blood pressure of a user using a portable electronic device. The method includes: receiving a measurement trigger signal by a control processor of the portable electronic device to perform a measurement routine to measure a blood pressure of a user; executing the measurement routine by the control processor responsive to the measurement trigger signal by: directing projecting of illumination through a body part that includes at least one elastic blood circulatory pathway through which flows a continuously changing volume of blood, the illumination being at a frequency absorbed by blood; directing generating of a detection output signal responsive to receiving portions of the illumination passing through the body part without absorption or reflection, such that the detection output signal corresponds to the continuously changing volume of blood; directing monitoring a holding force being applied to the body part during the directing projecting and the directing generating; and generating a blood pressure output signal based on the detection output signal and calibration data, the calibration data associating blood volume to blood pressure for the user and accounting for the holding force.

### BRIEF DESCRIPTION OF THE DRAWINGS

The accompanying drawings, referred to herein and constituting a part hereof, illustrate embodiments of the disclosure. The drawings together with the description serve to explain the principles of the invention.

FIG. 1 shows a block diagram of a portable electronic device, according to various embodiments described herein.

FIG. 2 shows an example blood pressure measurement environment including components of an illustrative portable electronic device.

FIG. 3 shows simplified plots of an illustrative detection output signal and a corresponding illustrative blood pressure output signal.

FIGS. 4A-4C show illustrative configuration use cases including embodiments of portable electronic devices having different portable housing implementations.

FIG. 5 shows a flow diagram of an illustrative method for operation of a portable device for non-invasive cuff-less measurement of blood pressure, according to various embodiments described herein.

FIG. 6 shows a flow diagram of a method for non-invasive cuff-less measurement of blood pressure of a user using a portable electronic device, according to various embodiments described herein.

FIG. 7 shows a flow diagram of a method for calibration of a portable electronic device for use in non-invasive cuff-less measurement of blood pressure of a user, according to various embodiments described herein.

FIG. 8 shows an example of a user with an approximate location of the heart indicated by location.

In the appended figures, similar components and/or features can have the same reference label. Further, various components of the same type can be distinguished by following the reference label by a second label that distinguishes among the similar components. If only the first reference label is used in the specification, the description is applicable to any one of the similar components having the same first reference label irrespective of the second reference label.

### DETAILED DESCRIPTION

In the following description, numerous specific details are provided for a thorough understanding of the present invention. However, it should be appreciated by those of skill in the art that the present invention may be realized without one or more of these details. In other examples, features and techniques known in the art will not be described for purposes of brevity.

It has become commonplace for individuals to use smartphones, smart watches, and/or other portable (e.g., wearable) electronic devices to obtain vital signs and related measurements. For example, many conventional wearable fitness trackers record a wearer's body temperature, heart rate, etc. to help the user monitor general health conditions, exercise, sleep, ad/or the like. While it can also be desirable to monitor blood pressure, conventional portable electronic devices and related techniques have not tended to be able to accurately measure blood pressure.

Conventionally, non-invasive blood pressure measurement is performed using a sphygmomanometer, which includes an inflatable blood pressure cuff connected to a inflation pump and a pressure gauge. The cuff is evenly tightened around the upper arm of a person, and a stethoscope disk is placed on the inner side of the upper arm under the cuff. The cuff is then pumped up rapidly until the gauge on the cuff shows a high reading (e.g., considerably higher than the person's typical systolic blood pressure), indicating blood flow has been occluded in the region of the cuff. Air is slowly let out of the cuff until a heart sound is first heard in the stethoscope, at which point the gauge reading is recorded as the person's systolic blood pressure. Air continues to be let out until the heart sounds are no longer heard through the stethoscope, at which point the gauge reading is recorded as the person's diastolic blood pressure. At any particular measurement location in a body (e.g., at the upper arm), the measured blood pressure can relate to the heart pump strength, the blood flow resistance, and the altitude and gravity difference between the measurement location and the heart. For example, when the heart is relaxed, blood flows relatively slowly throughout the body at relatively low pressure; when the heart contracts, a resulting pressure wave quickly travels through the blood throughout the circulatory system. In the conventional cuff-based approach to blood pressure measurement, the chosen measurement location has a relatively large amount of blood flow and is generally close to the same altitude (and gravity) as the heart. The systolic blood pressure then indicates the maximum pressure of the pressure wave in the blood stream caused by the contraction of the heart (i.e., during a systole), and the diastolic blood pressure indicates the minimum pressure while the heart is relaxing and refilling with blood (i.e., during a diastole).

While cuff-based blood pressure measurement procedures can tend to provide accurate measurements of both systolic and diastolic blood pressure, they tend to have various limitations. One limitation of such a cuff-based procedure is the reliance on various pieces of specialized equipment, including a sphygmomanometer and a stethoscope, which tend to be large, bulky, expensive, and not readily available to most individuals. Another limitation of such a cuff-based procedure is the reliance on active manual engagement with the measurement equipment by one or more individuals. Another limitation of such a cuff-based procedure is that obtaining accurate measurements tends to involve spending sufficient time and having sufficient focus to slowly release pressure in the cuff, listen for heart sounds, and accurately record systolic and diastolic readings. Another limitation of such a cuff-based procedure is that individuals with limited coordination, dexterity, hearing, etc. may be unable to perform such procedures.

Another conventional non-invasive approach to blood pressure measurement is based on previously studied and calibrated relationships between blood pressure and pulse transit time (PTT). PTT-based approaches have been thoroughly tested and used over at least the past decade, but such approaches also have various limitations. One such limitation is that PTT-based approaches typically rely on concurrent collection of data from at least two different sensors in two locations on a person's body, such as by placing an electrocardiogram (ECG) sensor at a person's heart and a pulse detector at a peripheral location (e.g., a fingertip). A related limitation is that obtaining a blood pressure measurement can then involve concurrently monitoring, synchronizing (e.g., including accurate peak detection), analyzing, and/or otherwise processing the signals from both sensors.

In an attempt to use more portable and accessible types of measurement devices, some PPT-based and other types of non-invasive blood pressure measurement approaches have sought to use so-called photoplethysmography (PPG). PPG-based approaches typically involve placing a light source (e.g., an infrared light-emitting diode) and light detector on a same side of a fingertip, or other body part. Materials in the blood (e.g., hemoglobin) tend to absorb certain wavelengths of light that do not tend to be absorbed (or not to the same degree) by surrounding tissue. Exploiting that concept, light from the light source is projected into the fingertip; portions of light tend to be absorbed, transmitted, or reflected as it interacts with blood and other biological features; and reflected portions of the light are detected by the light detector to produce a received reflection signal. The received reflection signal is recorded over time (e.g., over one to two minutes) to generate a heartbeat signal profile that indicates a pulse.

Technically, a pulse rate is a rate of increases (pulses) in palpable blood pressure throughout the body, which synchronizes and correlates to heart rate in a healthy individual. As such, while pulse measurements relate to the timing of changes in blood pressure, the pulse measurement do not provide a measure of the blood pressure itself (i.e., the systolic and/or diastolic blood pressure measurements). Indeed, PPG-based techniques have been successfully used in various contexts as pulse sensors (e.g., in smart watches, etc.), but such techniques have not tended to be able to provide accurate blood pressure measurements for a number of reasons. One reason is that the light path in PPG implementations, from the light source to the light detector, tends to intersect with different networks of arteries, vessels, capillaries, etc., such that the received reflection signal can tend to be a noisy signal, and slight changes in location of the light source and/or light sensor can result in different signal information. Another reason is that, even in laboratory-based, or other carefully controlled PPG implementations that yield relatively clean heartbeat signal profiles (i.e., received reflection signals) with relatively clear systolic peaks, the diastolic peaks (or troughs) can be difficult to accurately detect. For example, the diastolic blood flow can be relatively slow-moving and low-pressure, which can make it difficult to obtain useful measurement data, especially in regions with relatively small blood volume, such as in a fingertip. As such, even if peak-to-peak measurements between the systolic peaks can yield useful heart rate or pulse information, there typically remains insufficient data for accurate blood pressure measurements.

Another reason is that PPG-based implementations typically cannot account for certain factors, such as pressure on the blood flow caused by the PPG-based measurement device itself, and/or location of the body part relative to the heart, which can have appreciable impacts on characteristics of the received reflection signal. As noted above, at any particular measurement location in a body, the measured blood pressure can relate to the heart pump strength, the blood flow resistance, and the altitude and gravity difference between the measurement location and the heart. Thus, any pressure magnitude information obtained by PPG-based devices can be appreciably impacted by the selection of measurement location (e.g., even if a same user takes a measurement at a fingertip while his arm is hanging by his side versus while his arm is bent), the amount of pressure being exerted on the measurement location by the device itself (e.g., if the device is clamped onto the fingertip, or in a watch with a watchband cinched around a wrist), and/or other factors.

Embodiments described herein include a novel approach to non-invasive cuff-less blood pressure measurement based on using optical techniques to measure oscillations between systolic and diastolic blood volume in a body part. Generally, increases in blood volume tend to increase central venous pressure corresponding to an increase in right atrial pressure in the heart. With the increase in right atrial pressure comes a corresponding increase in right ventricular preload (i.e., end-diastolic pressure) and right-side stroke volume. This causes a corresponding increase in blood flow to the left ventricular and a corresponding increase in left ventricular preload and left-side stroke volume. The overall result of the increased stroke volume of the heart is an increase in cardiac output and blood pressure at the output of the heart. As described herein, calibration techniques are used to establish deterministic mappings between blood volume measurements and corresponding blood pressure readings. Embodiments can also account for selection of measurement location, amount of pressure being exerted on the measurement location by the device itself, and/or other factors.

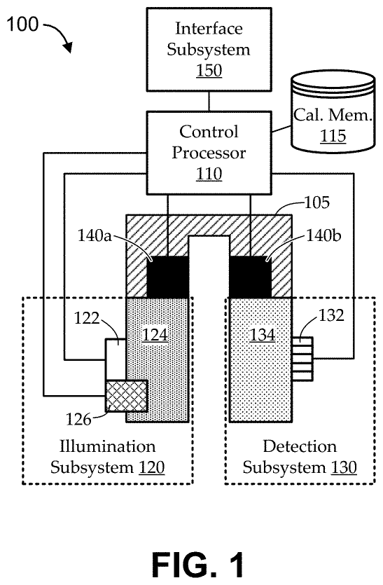

Turning to FIG. 1, a block diagram is shown of a portable electronic device 100, according to various embodiments described herein. The portable electronic device 100 is an implementation of a system for non-invasive, cuff-less measurement of blood pressure of a user. As described above, a contraction of the heart muscles (during a systole) causes a pressure wave to travel through the blood throughout the various elastic blood circulatory pathways of the circulatory system, such as in arteries, veins, capillaries, etc. The magnitude of pressure of the pressure wave can be different at different check points in the circulatory system (i.e., corresponding to different body parts) based on the sizes and types of elastic blood circulatory pathways in that check point, the location of the check point relative to the heart, restrictions on blood flow to that check point, etc. Thus, embodiments of the portable electronic device 100 are configured to be placed and/or held in contact with one or more body parts of the user that include at least one such elastic blood circulatory pathway through which flows a continuously changing volume of blood. In general, it can be preferable to select the body part to have good (e.g., relatively uniform) distribution of elastic circulatory pathways, while minimizing presence of tissues other than those elastic circulatory pathways. It can also be preferable for the design of the portable electronic device 100 to facilitate easy placement of the sensor components on desired body part locations and to facilitate consistent sensing location and conditions over multiple sessions. During a calibration routine, such placement of the portable electronic device 100 permits calibration of the portable electronic device 100 to the body part, the user, etc. During a measurement routine, such placement permits obtaining of blood pressure measurements based on optically detecting the changing volume of blood in the body part and based on the calibration. For example, as described more fully below, a portable housing 105 of the portable electronic device 100 can be configured to be slid or clamped onto a fingertip, earlobe, or wrist; held against a stomach, forehead, or neck; etc.

As illustrated, the portable electronic device 100 includes a control processor 110, an illumination subsystem 120, an optical detection subsystem 130, and the portable housing 105. Some implementations include additional components, such as one or more of a pressure detection subsystem 140, an interface subsystem 150, or a calibration memory 115. During use, a body part (including at least one elastic blood circulatory pathway through which flows a continuously changing volume of blood, as described above) is placed between the illumination subsystem 120 and the optical detection subsystem 130.

The illumination subsystem 120 projects illumination through the body part at an optical frequency that is absorbed by blood. For example, various species of hemoglobin in blood tend to absorb light very well at wavelengths between around 550-600 nm. Embodiments of the illumination subsystem 120 can include a set of (i.e., one or more) illumination sources 122 each to project the illumination through the body part and toward the optical detection subsystem 130. For example, the set of illumination sources 122 includes one or more light-emitting diodes (LEDs) that output the illumination at one or more optical frequencies absorbed by blood.

Some embodiments of the illumination subsystem 120 further include an illumination spreader 124 to increase a uniformity of illumination distribution from the set of illumination sources 122 over an illumination region of the body part. In some implementations, the illumination spreader 124 includes a diffuser material. For example, the set of illumination sources 122 are point sources of illumination, and the diffuser material of the illumination spreader 124 spreads the point-source illumination into a diffuse illumination of the illumination region of the body part. In some such implementations, the diffuser material is a diffuser film, such as a transparent material film having micro-prisms, or other shapes patterned thereon, which tend to cause diffusion of light projected through the film. In other such implementations, the diffuser material includes waveguide elements, which effectively convert a small number of input illumination sources 122 into a larger number of output illumination sources that can be further spread out over the illumination region. In other implementations, the illumination spreader 124 includes an air gap over the illumination region of the body part, and the illumination sources 122 are configured to project illumination in a manner that generally diffuses over the air gap. For example, such an illumination spreader 124 can include one or more spacing structures that holds the illumination sources 122 some distance away from the illumination region of the body part. In some such implementations, the air gap can be surrounded by reflective material, or the like, so that illumination projected into the air gap tends to be reflected and multiplied around the air gap to diffusely fill the air gap with illumination. Embodiments of the illumination subsystem 120 can also include optical shielding components to mitigate impacts of ambient light on optical measurements. For example, the illumination spreader 124 can include an opaque frame.

Various embodiments of the illumination subsystem 120 can be optimized for certain features. For example, illumination sources 122 can be configured to project a probe beam of illumination so that blood can absorb a desired ratio of the probe beam energy, while other proximate tissues (bones, fat, nerves etc.) absorb little or none of the probe beam energy. The arrangement of illumination sources 122 and/or the illumination spreader 124 can be configured so that the illumination region is expanded to cover enough quantity of blood vessels and/or other elastic circulatory pathways. The illumination sources 122, illumination spreader 124, and or other components can be modulated to mitigate, or even eliminate environmental influences, such as ambient light. The illumination sources 122 and/or other active components can be designed for low power consumption.

Some embodiments of the illumination subsystem 120 include an illumination monitor 126 to monitor illumination output of the set of illumination sources 122. For example, the illumination monitor 126 can detect whether one of the set of illumination sources 122 is failing, or otherwise no longer projecting illumination; whether there is an appreciable change in brightness, color, or stability of the illumination; etc. In some implementations, the illumination monitor 126 includes one or more photo-sensors to directly monitor characteristics of the illumination being output by the illumination sources 122. In other implementations, the illumination monitor 126 indirectly monitors the illumination output of the illumination sources 122, such as by monitoring driving current through the illumination sources 122, and/or other electrical characteristics. For example, embodiments of the portable electronic device 100 can include a portable power source (e.g., a rechargeable battery) to drive the illumination subsystem 120, and a failure, low voltage condition, or the like can negatively impact illumination output of the illumination sources 122. Embodiments of the illumination monitor 126 can output one or more signals to indicate a present condition of the illumination output.

As noted above, the illumination subsystem 120 and the optical detection subsystem 130 are on either side of a body part during use. By locating the optical detection subsystem 130 on the opposite side of the body part, the optical detection subsystem 130 receives portions of the projected illumination that pass through the body part without absorption or reflection. Embodiments of the optical detection subsystem 130 can then generate a detection output signal based on the received portions of the illumination to correspond to the continuously changing volume of blood. Embodiments of the optical detection subsystem 130 can include a set of photodetectors 132 to generate the detection output signal in response to exposure to the received portions of the illumination. In some implementations, the set of photodetectors 132 is implemented as an array of detection pixels, as a charge-coupled device (CCD) array, or any other suitable configuration of photodetectors 132. Some implementations of the set of photodetectors 132 include readout circuitry to facilitate generation of the detection output signal. For example, the readout circuitry can include filters, amplifiers, analog-to-digital converters, and/or other suitable circuitry. In some embodiments, the optical detection subsystem 130 also includes a receiving aperture 134 to direct the received portions of the illumination onto the set of photodetectors 132. The receiving aperture 134 can include any suitable components to facilitate receipt of the portions of the illumination as incident illumination on the photodetectors 132. For example, the receiving aperture 134 can include lenses, shutters, filters, light-guides, etc. Some embodiments implement the optical detection subsystem 130 as an imaging system, such as an video imaging system having integrated optics, photodetector 132 array, supporting circuitry, etc.

Embodiments of the control processor 110 are in communication with at least the optical detection subsystem 130 to generate a blood pressure output signal based on the detection output signal and based on calibration data that associates blood volume to blood pressure for the portable electronic device 100 and/or the user. Embodiments of the control processor 110 can include a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction set computer (RISC) processor, a complex instruction set computer (CISC) processor, a microprocessor, or the like, or any combination thereof. The control processor 110 can be in communication with a memory (e.g., the calibration memory 115 described below, instruction memory, etc.), which can include at least non-transient storage for providing processor-readable instructions to the control processor 110 and for storing various types of data to support features described herein. In some embodiments, such memory is all local storage (e.g., one or more solid-state drives, hard disk drives, registers, etc.) of the portable electronic device 100. Additionally or alternatively, embodiments of such a memory can include remote storage (e.g., a remote server), distributed storage (e.g., cloud-based storage), or other non-local storage.

In some embodiments, the optical detection subsystem 130 outputs the detection signal to the control processor 110 as a digital signal. In other embodiments, the optical detection subsystem 130 outputs the detection signal to the control processor 110 as an analog signal, and the control processor 110 includes an analog-to-digital conversion stage. In general, it can be expected that pumping of the heart will generate substantially periodic pulse waves in the blood flowing through the arteries, or other elastic circulatory pathways of the body part. Movement of the pulse waves through the circularity pathways of the body part will correspondingly cause substantially periodic increases and decreases in blood volume relating to the magnitude of pressure change during the presence and absence of the pulse wave. This blood volume change will correspondingly cause substantially periodic increases and decreases in the amount of projected illumination being absorbed by the local blood volume, thereby correspondingly causing substantially periodic decreases and increases in the amount of illumination that passes through to the optical detection subsystem 130 without absorption or reflection.

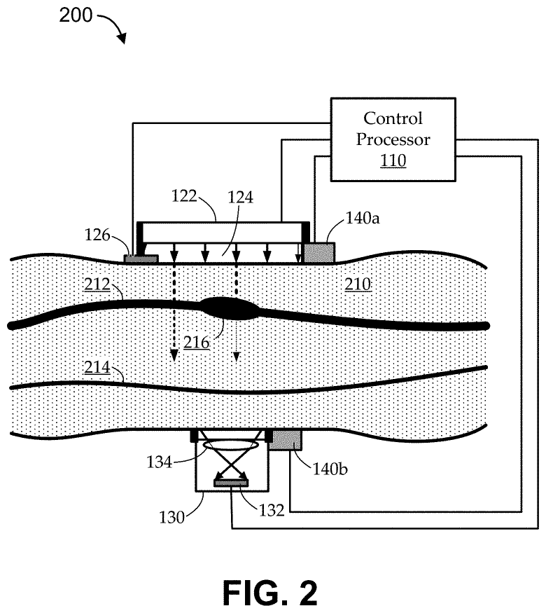

For the sake of illustration, FIG. 2 shows an example blood pressure measurement environment 200 including components of an illustrative portable electronic device 100. As illustrated, during use, the portable electronic device 100 is positioned relative to a body part 210, such that components of the illumination subsystem 120 are on one side of the body part 210, and components of the optical detection subsystem 130 are on an opposite side of the body part 210. In particular, on one side of the body part 210, a set of illumination sources 122 are shown generally projecting light into the body part 210, through an illumination spreader 124 implemented as an air gap, generally along directions indicated by arrows. On the same side of the body part 210, the illustrative configuration includes an illumination monitor 126. On the other side of the body part 210, the configuration is shown to include a receiving aperture 134 (e.g., including a lens and/or other optics) to direct received illumination onto a set of photodetectors 132. The illustrated configuration also shows the pressure detection subsystem 140 implemented as components on either side of the body part 210, such as a pair of force sensors. All of the illustrated components are shown coupled with (e.g., in electrical communication with) the control processor 110. Structures of the portable housing 105 and other components are not shown to avoid overcomplicating the Figure.

The body part 210 includes various elastic circulatory system pathways, such as arteries 212, veins 214, etc. As describe above, it can be expected that pumping of the heart will generate substantially periodic pulse waves in the blood flowing through the elastic circulatory pathways, which will cause corresponding periodic changes in blood volume. An exaggerated and simplified example of a transient change in blood volume resulting from a traveling pulse wave in the blood stream is illustrated by reference 216. In fact, the body quickly and efficiently adapts to such changes in blood volume and pressure, such that the transient changes may be practically undetectable (or too small to provide any reliable measurement data) in most elastic pathways of the circulatory system other than main arteries (e.g., the volume change is shown only in the artery 212 in FIG. 2). It can be seen that portions of the illumination passing through regions of lower blood volume are less attenuated (i.e., less absorbed) than portions of the illumination passing through regions of higher blood volume. As such, changes in the level of illumination as received by the optical detection subsystem 130 can correspond to changes in the blood volume caused by the passing pulse wave.

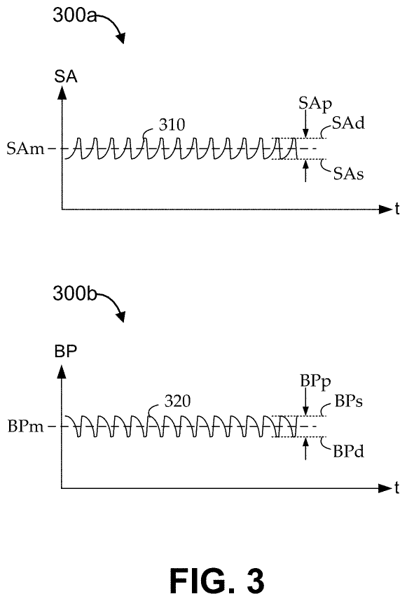

For further clarity, FIG. 3 shows simplified plots 300 of an illustrative detection output signal 310 and a corresponding illustrative blood pressure output signal 320. The detection output signal 310 illustrates a measured detection signal output magnitude (SA) over time, and the blood pressure output signal 320 illustrates a derived blood pressure magnitude (BP) over time. Both the detection output signal 310 and the blood pressure output signal 320 are illustrated as periodic signals. Each time a pressure wave passes through the elastic circulatory pathways of the body part, there are corresponding, generally periodic increases and decreases in blood volume in those pathways. The generally periodic changes in blood volume cause generally periodic decreases and increases in the amount of illumination reaching the optical detection subsystem 130, which manifests as generally periodic decreases an increases in SA in the detection output signal 310. In particular, each pressure wave manifests as a pulse in the detection output signal 310 having a peak-to-peak amplitude (SAp) that is effectively the difference between a diastolic peak signal level (SAd) and a systolic trough signal level (SAs). Over time, the pulses of the detection output signal 310 also manifest a mean signal amplitude (SAm). In some implementations, the SAm can be used to normalize the SAd and SAs information, and/or to support other data processing features.

The control processor 110 can generate the blood pressure output signal 320 based on this detection output signal 310. As shown in plot 300b, each pressure wave ultimately manifests as a pulse in the blood pressure output signal 320 having a peak-to-peak amplitude (BPp) that is effectively the difference between a systolic peak level (BPs) and a diastolic trough level (BPd). As noted above, SA corresponds directly to blood volume changes as manifest by received illumination levels, but SA does not directly correspond to blood pressure measurements. Instead, the control processor 110 uses calibration data to convert SA data from the detection output signal 310 into BP data for the blood pressure output signal 320. In some implementations, the calibration data includes a lookup table of SA values and corresponding BP values obtained during a prior calibration routine. In other implementations, the calibration data includes weighting factors, and/or other values to apply to a formulaic mapping of SA to BP values. In some embodiments, the control processor 110 generates the blood pressure output signal 320 as a continuous (e.g., digital) signal, such as illustrated in plot 300b, to include a periodic sequence of pulses with corresponding BPp, BPs, BPd, and BPm. In other embodiments, additionally or alternatively, the control processor 110 generates the blood pressure output signal 320 as a set of blood pressure measurement data. For example, embodiments of the control processor 110 can apply statistical processing techniques, sliding window techniques, and/or other processing techniques to the detection output signal 310 to compute a single value for SAd and a single value for SAs; and the single SAd and SAs values can be converted to BPd and BPs values based on the calibration data.

Returning to FIG. 1, embodiments of the control processor 110 can include, or be in communication with, a calibration memory 115 to store the calibration data. The calibration memory 115 can include any suitable type of memory, such as registers, solid state memory, etc., and the calibration data can be stored in any suitable manner. For example, as noted above, the calibration data can be stored as a lookup table, as a set of coefficients or weighting factors, etc.

Embodiments of the control processor 110 can be in communication with other components of the portable electronic device 100, such as with the illumination subsystem 120. In such embodiments, the control processor 110 can direct operation of the illumination subsystem 120 and the optical detection subsystem 130 during the measurement routine and/or during the calibration routine. In some embodiments, during such a routine, the control processor 110 directs the illumination subsystem 120 to turn on illumination sources to project illumination though the body part, and the control processor 110 monitors signals from the illumination monitor 126 to determine whether the illumination output from the illumination sources 122 is satisfying predetermined acceptance criteria (e.g., that each illumination source 122 appears to be operating properly, that at least a predetermined threshold brightness of illumination is being output by the illumination sources 122, that the level of illumination being output by the illumination sources 122 is varying by less than a predetermined threshold amount over time, etc.).

Embodiments of the control processor 110 can also be in communication with a pressure detection subsystem 140. The pressure and volume of blood reaching a particular location in the circulatory system can be impacted by external restrictions to the blood flow. For example, when the portable housing 105 is clamped or pressed against the body part being used as the blood pressure check point, a holding force (e.g., clamping force, pressing force, constricting force, etc.) being exerted by the portable housing 105 on the body part can impact the measurement of blood pressure at that location. A pressure detection subsystem 140 can be integrated with the portable housing 105 to monitor the holding force present during the measurement routine (e.g., and during the calibration routine). Embodiments of the pressure detection subsystem 140 can include one or more sensors to measure the holding force. Depending on the configuration of the portable housing 105 and/or the manner of holding the portable housing 105 against the body part, different configurations and/or types of the sensors of the pressure detection subsystem 140 can be used. In some implementations, one or more force sensors are used to detect the holding force directly. In other implementations, sensors of the pressure detection subsystem 140 indirectly detect the holding force, such as by detecting a distance between the illumination subsystem 120 and the body part, or the like (e.g., less distance can be assumed to correspond to more holding force, etc.).

In some embodiments, the calibration data (e.g., stored in the calibration memory 115) further accounts for measurements of holding force. While the portable electronic device 100 is being used to obtain blood pressure measurements with the illumination subsystem 120 and the optical detection subsystem 130, the holding force can also be monitored by the pressure detection subsystem 140 and recorded by the control processor 110. In some implementations, a dynamic feedback control loop is used to direct the user to apply a particular amount of holding force (e.g., via human-discernable feedback provided via the interface subsystem 150, as described below). For example, the control processor 110 uses such dynamic feedback to direct the user to configure the portable electronic device 100 in such a manner that substantially the same amount of holding force is being presently applied as was previously applied during a calibration routine. In other implementations, the pressure detection subsystem 140 monitors the holding force to determine whether a predetermined threshold amount of holding force is exceeded. For example, in relation to certain body parts, it can be determined that only a holding force in excess of the predetermined threshold level will have a measurable impact on blood flow in that region. In other implementations, the calibration data is multidimensional, such that any detected particular detection output signal level are calibrated as corresponding to different blood pressure output signal levels, depending on a concurrently detected holding force.

In some embodiments, the portable electronic device 100 further includes an interface subsystem 150. In some implementations, the interface subsystem 150 includes one or more wired and/or wireless ports by which to provide one or more wired and/or wireless communication interfaces between the portable electronic device 100 and an external system. For example, the interface subsystem 150 can include a one or more wired or wireless network interfaces, such as for communicating with wireless fidelity (WiFi), Bluetooth, ZigBee, cellular, satellite, Ethernet, cable, and/or any other suitable wired or wireless communication network; one or more peripheral interfaces, such as for interfacing with a headphone jack, a display port, etc.; etc. In other embodiments, the interface subsystem 150 includes an integrated human-readable display (e.g., one or more indicator LEDs, a touchscreen display, a LED display, a liquid crystal display (LCD), etc.) by which to visually provide human-discernable information to a user. In other embodiments, the interface subsystem 150 additionally or alternatively includes one or more audio transducers by which to provide human-discernable audio output. In various embodiments, the human-discernable information can include graphical output (e.g., alphanumeric, images, illumination of an indicator, etc.), audible output (e.g., synthesized sounds and/or speech, recorded sounds and/or speech, etc.), haptic output (e.g., vibration, etc.), etc. Some implementations additionally or alternatively generate machine-discernable information, such as for use by other computational devices to generate further analyses, provide human-discernable outputs, etc.

For example, as described above, some embodiments of the control processor 110 computes the blood pressure output signal as a waveform (e.g., as in plot 200b of FIG. 2). Additionally or alternatively, generating the blood pressure output signal by the control processor 110 can include generating (e.g., computing) a systolic blood pressure reading and a diastolic blood pressure reading. One or both of these readings can be output in any suitable manner via the interface subsystem 150. In some embodiments, a graphical representation of the blood pressure output signal waveform and/or blood pressure readings is displayed via an integrated display of the interface subsystem 150. In other embodiments, a graphical, audio, or other representation of the blood pressure output signal waveform and/or blood pressure readings is output via an integrated output interface and/or via an external interface in communication with the portable electronic device 100 via the interface subsystem 150.

In some embodiments, the interface subsystem 150 includes one or more input interfaces, such as to receive information from a user, from one or more peripheral devices, and/or from other computational systems. Such input interfaces can include one or more buttons, keypads, ports, touchscreen interfaces, etc. In some implementations, interface buttons can be used to selectively place the portable electronic device 100 into a measurement mode (for performing the measurement routine), or into a calibration mode (for performing the calibration routine). In some implementations, such input interfaces of the interface subsystem 150 are sued to support receipt of certain data during the calibration routine. For example, calibration of the portable electronic device 100 can involve concurrently obtaining measurements by the portable electronic device 100 and by a pre-calibrated blood pressure measurement device, such as a sphygmomanometer. In some such cases, measurements collected by the pre-calibrated blood pressure measurement device can be manually input to the portable electronic device 100 via interface elements of the interface subsystem 150 (e.g., a touchscreen, keypad, etc.). For example, during the calibration routine, the control processor 110 directs the interface subsystem 150 to output a human-discernable prompt for manual entry of measurement values collected by the pre-calibrated blood pressure measurement device. In other such cases, the pre-calibrated blood pressure measurement device generates a machine-discernable output, which can be communicated directly to the portable electronic device 100 via wired or wireless ports of the interface subsystem 150.

Embodiments of the portable housing 105 can be configured to house some or all components of the portable electronic device 100. Some embodiments of the portable housing 105 are structurally configured to hold at least one of the illumination subsystem or the optical detection subsystem against the body part with a holding force during the measurement routine and/or the calibration routine. Some embodiments of the portable housing 105 are further configured to fully house the control processor 110 within the portable housing 105. Some embodiments of the portable housing 105 also have the pressure detection subsystem 140 integrated therein. Some embodiments of the portable housing 105 also have some or all of the interface subsystem 150 integrated therein, such as including one or more integrated displays, integrated buttons, integrated ports, etc.

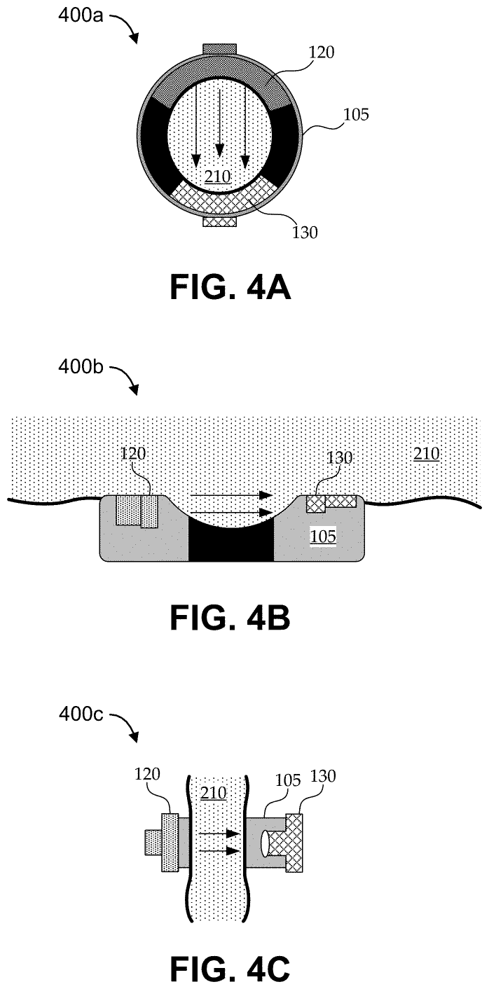

In various embodiments, the portable housing 105 is configured to hold the illumination subsystem 120 on a first side of the body part, to hold the optical detection subsystem 130 on a second side of the body part opposite the first side, and to orient the illumination subsystem 120 to project the illumination through the body part at least in a direction of the optical detection subsystem 130. For example, FIGS. 4A-4C show illustrative configuration use cases 400 including embodiments of portable electronic devices 100 having different portable housing 105 implementations. In FIG. 4A, the portable housing 105 is implemented as a ring-shaped structure configured to fully surround a small body part 210. For example, the body part 210 is a finger, a toe, etc. With the body part 210 situated within the ring-shaped portable housing 105, the illumination subsystem 120 is held on the first side of the body part 210, the optical detection subsystem 130 is held on the second side of the body part 210 opposite the first side, and the illumination subsystem 120 is oriented to project the illumination through the body part 210 at least in the direction of the optical detection subsystem 130.

In FIG. 4B, the portable housing 105 is implemented as a half-open-shaped structure configured to partially surround a body part 210. For example, the body part 210 is a stomach, forehead, wrist, etc. Holding or pressing the portable housing 105 against the body part 210 positions the body part 210 so that the illumination subsystem 120 is on the first side of the body part 210, the optical detection subsystem 130 is on the second side of the body part 210 opposite the first side, and the illumination subsystem 120 is oriented to project the illumination through the body part 210 at least in the direction of the optical detection subsystem 130. Notably, even though the portable housing 105 only partially surrounds the body part 210, illumination is being projected through the body part 210, such that the illumination received by the optical detection subsystem 130 is the portion of the illumination that has passed through the body part without having been absorbed or reflected away by the blood in the body part (i.e., by the blood flowing through the arteries, veins, etc. in the body part 210).

In FIG. 4C, the portable housing 105 is implemented as a clip-shaped structure configured to clip onto a body part 210. For example, the body part is an ear lobe, fingertip, toe tip, etc. Clipping the portable housing 105 onto the body part 210 positions the body part 210 so that the illumination subsystem 120 is on the first side of the body part 210, the optical detection subsystem 130 is on the second side of the body part 210 opposite the first side, and the illumination subsystem 120 is oriented to project the illumination through the body part 210 at least in the direction of the optical detection subsystem 130.

In some embodiments, the portable electronic device 100 is implemented in one or more separated housing structures that can be in communication via wired and/or wireless communications. As one example, any of the configuration use cases 400 of FIGS. 4A-4C can be implemented as a single portable housing 105 that houses all of the components of the portable electronic device 100, including an integrated interface subsystem 150 that includes an integrated touchscreen display interface. Alternatively, any of the configuration use cases 400 of FIGS. 4A-4C can be implemented as two separate housings: a first portable housing 105 that houses the illumination subsystem 120, the optical detection subsystem 130, and the pressure detection subsystem 140; and a second portable housing, coupled with the first portable housing 100, and including a battery, the control processor 110, the calibration memory 115, and the interface subsystem 150 with the integrated touchscreen display interface. Alternatively, any of the configuration use cases 400 of FIGS. 4A-4C can be implemented for coordination with one or more additional computational platforms. For example, the portable housing 105 includes the illumination subsystem 120, the optical detection subsystem 130, the pressure detection subsystem 140, the control processor 110, the calibration memory 115, and the interface subsystem 150; and the interface subsystem is configured for bi-directional communication with a smartphone, smart watch, fitness tracker, or the like, which includes a touchscreen display interface by which to control components in the portable housing 105 and to view measurement results and/or other information received from components of the portable housing 105.

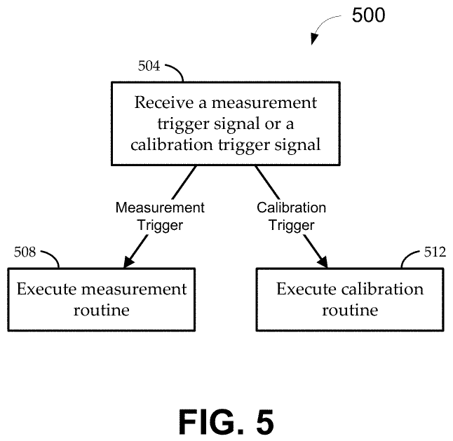

FIG. 5 shows a flow diagram of an illustrative method 500 for operation of a portable device for non-invasive cuff-less measurement of blood pressure, according to various embodiments described herein. Embodiments can begin by receiving a trigger signal at stage 504 indicating either to trigger performance of a measurement routine or to trigger performance of a calibration routine. For example, a control processor of the portable electronic device receives either a measurement trigger signal or a calibration trigger signal. In some implementations, receiving the trigger signal at stage 504 can include receiving an explicit user request via an interface component, such as a button, touchscreen interface, gesture interface, audio interface, etc. In other implementations, the device can be configured to perform the measurement and/or calibration routine according to a stored schedule (e.g., every few hours, once per day, etc.), and receiving the trigger at stage 504 includes receiving a signal from a scheduling component. In other implementations, the device can be configured to perform the measurement and/or calibration routine according to a detection event (e.g., detecting a large change in heart rate, body temperature, etc.), and receiving the trigger at stage 504 includes receiving a signal from a detection component. In any of these and/or other implementations, the method can continue either by executing the measurement routine at stage 508 responsive to receiving a measurement trigger signal at stage 504, or by executing the calibration routine at stage 512 responsive to receiving a calibration trigger signal at stage 504.

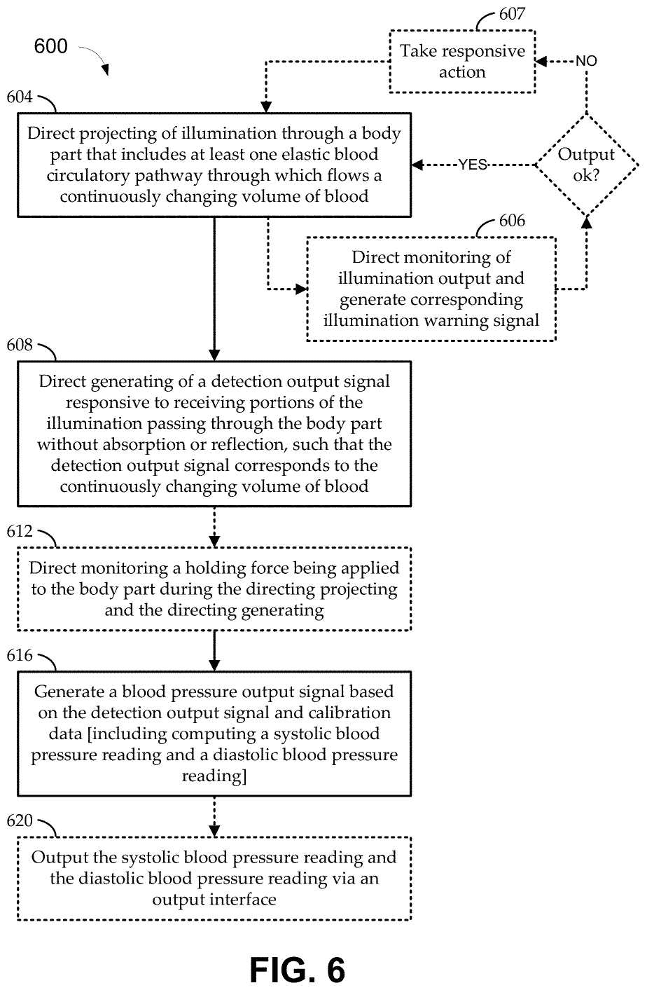

FIG. 6 shows a flow diagram of a method 600 for non-invasive cuff-less measurement of blood pressure of a user using a portable electronic device, according to various embodiments described herein. The method 600 can be an implementation the measurement routine of stage 508 of the method 500 of FIG. 5. Embodiments of the method 600 (i.e., of the measurement routine) can begin at stage 604 by directing projecting of illumination through a body part that includes at least one elastic blood circulatory pathway through which flows a continuously changing volume of blood. As described herein, the illumination is at a frequency absorbed by blood. At stage 608, embodiments can direct generating of a detection output signal responsive to receiving portions of the illumination passing through the body part without absorption or reflection, such that the detection output signal corresponds to the continuously changing volume of blood. At stage 612, some embodiments can direct monitoring a holding force being applied to the body part during the directing projecting and the directing generating. Some embodiments, during the illuminating at stage 604, can direct monitoring at stage 606 of an illumination output being projected through the body part and can generate an illumination warning signal responsive to the directing monitoring indicating whether the illumination output fails to satisfy a predetermined acceptance criteria. In response to detecting a failure to satisfy the predetermined acceptance criteria, some such embodiments can take responsive action at stage 607. For example, the responsive action can include stopping execution of the method 600, outputting a human-discernable indication of the illumination warning signal, attempting to restore proper functioning of the illumination sources 122 and/or other components, running a health check routine, and/or other responsive action.

At stage 616, embodiments can generate a blood pressure output signal based on the detection output signal and calibration data. As described above, the calibration data associates blood volume to blood pressure for the user. In some cases, the calibration data also accounts for the holding force monitored in stage 612. In some embodiments, the generating of the blood pressure output signal at stage 616 includes computing a systolic blood pressure reading and a diastolic blood pressure reading. Such embodiments can further include outputting the systolic blood pressure reading and the diastolic blood pressure reading via an output interface at stage 620.

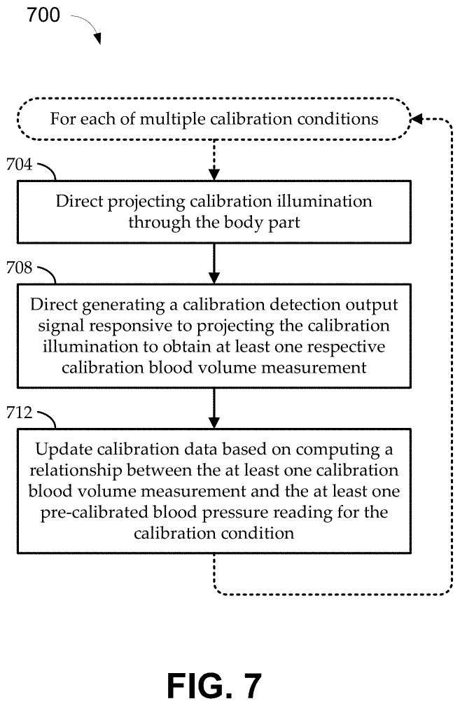

FIG. 7 shows a flow diagram of a method 700 for calibration of a portable electronic device for use in non-invasive cuff-less measurement of blood pressure of a user, according to various embodiments described herein. The method 700 can be an implementation of stage 512 of the method 500 of FIG. 5. In some embodiments, calibration is performed for each of a number of calibration conditions that correspond to respective placements of the portable electronic device. For example, the contraction of the heart muscles during systole provide sufficient pressure to move blood throughout the circulatory system, including all the way to the top of the head and all the way to the bottom of the feet. Reaching those different locations can involve overcoming different amounts of gravity and/or other forces. As such, a blood pressure measurement taken at any particular check point in the body can be highly dependent on the position of that check point relative to the heart.

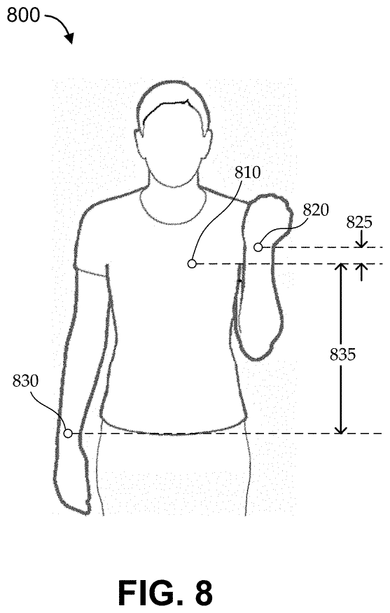

For the sake of illustration, FIG. 8 shows an example of a user 800 with an approximate location of the heart indicated by location 810. In the illustrated scenario, the user 800 is measuring blood pressure by placing the portable electronic device at his wrist. In one case, his arm is bent upward, thereby placing the portable electronic device at location 820. In another case, his arm is extended downward, thereby placing the portable electronic device at location 830. It can be seen that location 820 is relatively close to the heart location 810 with respect to gravitational forces (illustrated by distance 825), while location 830 is relatively far from the heart location 810 with respect to gravitational forces (illustrated by distance 835). Some embodiments of the calibration routine can involve obtaining calibration data with the wrist in each of locations 820 and 830. For example, placing the wrist in position 820 may provide relatively good data for calibration of diastolic blood pressure measurement, and placing the wrist in position 830 may provide relatively good data for calibration of systolic blood pressure measurement. Similarly, measurements taken at the different locations can be used to obtain a mean blood pressure level, and/or other statistical measurements. Other embodiments of the calibration routine can use more than two locations, different locations, etc.

Returning to FIG. 7, embodiments of the calibration routine assume that blood pressure measurements are being obtained concurrently by both the portable electronic device and by a pre-calibrated blood pressure measurement device, such as a sphygmomanometer. Some embodiments of the calibration routine can be performed in substantially the same manner as some or all of stages 604-612 of FIG. 6, except that blood pressure measurements are concurrently being obtained by the pre-calibrated blood pressure measurement device. For example, embodiments can begin at stage 704 by directing projecting calibration illumination through the body part. At stage 708, embodiments can direct generating a calibration detection output signal responsive to projecting the calibration illumination to obtain at least one respective calibration blood volume measurement. At stage 712, embodiments can update the calibration data (e.g., store new data, overwrite existing data, etc.) based on computing a relationship between the at least one calibration blood volume measurement and the at least one pre-calibrated blood pressure reading for the calibration condition.

While this disclosure contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

A recitation of “a”, “an” or “the” is intended to mean “one or more” unless specifically indicated to the contrary. Ranges may be expressed herein as from “about” one specified value, and/or to “about” another specified value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. When such a range is expressed, another embodiment includes from the one specific value and/or to the other specified value. Similarly, when values are expressed as approximations, by use of the antecedent “about,” it will be understood that the specified value forms another embodiment. It will be further understood that the endpoints of each of the ranges are included with the range.

All patents, patent applications, publications, and descriptions mentioned here are incorporated by reference in their entirety for all purposes. None is admitted to be prior art.

What is claimed is:

1. A system for non-invasive cuff-less measurement of blood pressure of a user, the system comprising:
an illumination subsystem to project illumination through a body part that includes at least one elastic blood circulatory pathway through which flows a continuously changing volume of blood, the illumination being at a frequency absorbed by blood;
an optical detection subsystem to receive portions of the illumination passing through the body part without absorption or reflection, and to generate a detection output signal based on the received portions of the illumination to correspond to the continuously changing volume of blood;
a portable housing by which to hold at least one of the illumination subsystem or the optical detection subsystem against the body part with a holding force during a measurement routine including illumination and optical detection;
a pressure detection subsystem integrated with the portable housing to monitor the holding force during the measurement routine; and
a control processor to generate a blood pressure output signal based on the detection output signal and calibration data, the calibration data associating blood volume to blood pressure for the user and accounting for the holding force.

2. The system of claim 1, further comprising:
an interface subsystem integrated with the portable housing and coupled with the control processor to output a systolic blood pressure reading and a diastolic blood pressure reading,
wherein generating the blood pressure output signal by the control processor comprises generating the systolic blood pressure reading and the diastolic blood pressure reading.

3. The system of claim 2, wherein the interface subsystem comprises a human-readable display to visually output the systolic blood pressure reading and the diastolic blood pressure reading.

4. The system of claim 1, further comprising:
a calibration memory having the calibration data stored thereon.

5. The system of claim 1, wherein the illumination subsystem comprises:
a set of illumination sources each to project the illumination through the body part and directed toward the detection subsystem; and
an illumination spreader to increase a uniformity of illumination distribution from the set of illumination sources over an illumination region of the body part.

6. The system of claim 5, wherein the illumination spreader comprises a diffuser material.

7. The system of claim 5, wherein the illumination spreader comprises:
a spacing structure to hold the set of illumination sources a distance away from the illumination region of the body part; and
a shielding structure to shield the illumination region of the body part from ambient illumination.

8. The system of claim 5, wherein:
the illumination subsystem further comprises an illumination monitor to monitor an illumination output of the set of illumination sources; and
the control processor is in communication with the illumination subsystem to generate an illumination warning signal responsive to the monitoring by the illumination monitor indicating that the illumination output fails to satisfy a predetermined acceptance criteria.

9. The system of claim 1, wherein the optical detection subsystem comprises:
a set of photodetectors to generate the detection output signal in response to exposure to the received portions of the illumination; and
a receiving aperture to direct the received portions of the illumination onto the set of photodetectors.

10. The system of claim 1, wherein the portable housing is to hold the illumination subsystem on a first side of the body part, to hold the optical detection subsystem on a second side of the body part opposite the first side, and to orient the illumination subsystem to project the illumination through the body part at least in a direction of the optical detection subsystem.

11. The system of claim 10, wherein the portable housing comprises a ring-shaped structure to fully surround the body part such that the illumination subsystem is held on the first side of the body part, the optical detection subsystem is held on the second side of the body part opposite the first side, and the illumination subsystem is oriented to project the illumination through the body part at least in the direction of the optical detection subsystem.

12. The system of claim 10, wherein the portable housing comprises a half-open-shaped structure to partially surround the body part such that the illumination subsystem is held on the first side of the body part, the optical detection subsystem is held on the second side of the body part opposite the first side, and the illumination subsystem is oriented to project the illumination through the body part at least in the direction of the optical detection subsystem.

13. The system of claim 10, wherein the portable housing comprises a clip-shaped structure to clip onto the body part such that the illumination subsystem is held on the first side of the body part, the optical detection subsystem is held on the second side of the body part opposite the first side, and the illumination subsystem is oriented to project the illumination through the body part at least in the direction of the optical detection subsystem.

14. The system of claim 1, wherein the control processor is integrated within the portable housing and is coupled with the illumination subsystem, the optical detection subsystem, and the pressure detection subsystem.

15. The system of claim 1, wherein the control processor is configured selectively to operate in one of a measurement mode or a calibration mode, such that:
in the measurement mode, the control processor is to perform the measurement routine to generate the blood pressure output signal based on the detection output signal and the calibration data; and
in the calibration mode, the control processor is to perform a calibration routine to generate and store the calibration data by, for each of a plurality of calibration conditions corresponding to a respective placement of the portable housing:
generating at least one detection output signal by the optical detection subsystem, responsive to projecting the illumination by the illumination subsystem with the portable housing in the respective placement, to obtain at least one respective calibration blood volume measurement;
obtaining at least one pre-calibrated blood pressure reading from a separate measurement device; and
updating the calibration data based on computing a relationship between the at least one calibration blood volume measurement to the at least one pre-calibrated blood pressure reading for the calibration condition.

16. The system of claim 15, wherein the control processor is to perform the calibration routine further by, for each of the plurality of calibration conditions:
obtaining a respective calibration holding force measurement from the pressure detection subsystem,
wherein the computing the relationship accounts for the respective calibration holding force measurement.

17. A method for non-invasive cuff-less measurement of blood pressure of a user using a portable electronic device, the method comprising:
receiving a measurement trigger signal by a control processor of the portable electronic device to perform a measurement routine to measure a blood pressure of a user;
executing the measurement routine by the control processor responsive to the measurement trigger signal by:
directing projecting of illumination through a body part that includes at least one elastic blood circulatory pathway through which flows a continuously changing volume of blood, the illumination being at a frequency absorbed by blood;
directing generating of a detection output signal responsive to receiving portions of the illumination passing through the body part without absorption or reflection, such that the detection output signal corresponds to the continuously changing volume of blood;
directing monitoring a holding force being applied to the body part during the directing projecting and the directing generating; and
generating a blood pressure output signal based on the detection output signal and calibration data, the calibration data associating blood volume to blood pressure for the user and accounting for the holding force.

18. The method of claim 17, wherein the generating the blood pressure output signal comprises computing a systolic blood pressure reading and a diastolic blood pressure reading, and the method further comprises:
outputting the systolic blood pressure reading and the diastolic blood pressure reading via an output interface.

19. The method of claim 17, further comprising:
directing monitoring of an illumination output being projected through the body part during the directing projecting; and
generating an illumination warning signal responsive to the directing monitoring indicating that the illumination output fails to satisfy a predetermined acceptance criteria.

20. The method of claim 17, further comprising:
receiving a calibration trigger signal by the control processor to perform a calibration routine to generate and store the calibration data for the portable electronic device and the user;
executing the calibration routine by the control processor responsive to the calibration trigger signal by, for each of a plurality of calibration conditions corresponding to a respective placement of the portable electronic device, while having a pre-calibrated blood pressure measurement device placed on the user to obtain at least one pre-calibrated blood pressure reading;
directing projecting calibration illumination through the body part;
directing generating a calibration detection output signal responsive to projecting the calibration illumination to obtain at least one respective calibration blood volume measurement; and
updating the calibration data based on computing a relationship between the at least one calibration blood volume measurement to the at least one pre-calibrated blood pressure reading for the calibration condition.
